(19) United States
(12) Patent Application Publication
(10) Pub. No.: US 2025/0339674 A1
(43) Pub. Date: Nov. 6, 2025

(54) ELECTROTHERAPY DEVICE

(71) Applicant: OMRON HEALTHCARE Co., Ltd., Kyoto (JP)

(72) Inventors: Tomoyuki NISHIDA, Kyoto (JP); Kazuya KITAYAMA, Kyoto (JP); Yoshiyuki TSUJI, Kyoto (JP)

(21) Appl. No.: 19/271,175

(22) Filed: Jul. 16, 2025

Related U.S. Application Data
(63) Continuation of application No. PCT/JP2023/040550, filed on Nov. 10, 2023.

(30) Foreign Application Priority Data
Mar. 14, 2023 (JP) 2023-039604

(51) Int. Cl. A61N 1/04

(52) U.S. Cl. CPC A61N 1/0476

(57) ABSTRACT

An electrical treatment device includes a main body portion that includes a first outer edge portion and a second outer edge portion facing each other in a first direction in a plan view, and a first electrode portion, and a second electrode portion. The first electrode portion and the second electrode portion are spaced apart from each other in the first direction and disposed to cause at least a central portion of the main body portion to be located therebetween. The first electrode portion includes a first peeling starting portion, and a first line located on a side of the second electrode portion. The second electrode portion includes a second peeling starting portion, and a second line located on a side of the first electrode portion and facing the first line in the first direction.

### CROSS-REFERENCE TO RELATED APPLICATIONS

This application is the U.S. national stage application filed pursuant to 35 U.S.C. 365(c) and 120 as a continuation of International Patent Application No. PCT/JP2023/040550, filed Nov. 10, 2023, which application claims priority to Japanese Patent Application No. 2023-039604, filed Mar. 14, 2023, which applications are incorporated herein by reference in their entireties.

### TECHNICAL FIELD

The present disclosure relates to an electrical treatment device.

### BACKGROUND ART

As a known electrical treatment device, JP 2017-189268 A (Patent Document 1) discloses a device including a pair of conductive adhesive sheets that are provided linearly with a space therebetween, and a treatment device main body (main body portion) provided straddling the pair of conductive adhesive sheets. A pinch tab is provided at an end portion of one of the conductive adhesive sheets on one side. When the electrical treatment device that has been attached to a treatment site is removed from the treatment site, the pinch tab is gripped, and the one conductive adhesive sheet provided with the pinch tab, the treatment device main body, and the other conductive adhesive sheet are linearly removed in this order from the treatment site.

### CITATION LIST

Patent Document 1: JP 2017-189268 A

### SUMMARY OF INVENTION

### Technical Problem

Since the electrical treatment device described in Patent Document 1 is peeled off using the pinch tab from the end portion side of the conductive adhesive sheet on the one side of the pair of conductive adhesive sheets, it is difficult to entirely remove the electrical treatment device from the treatment site. When the electrical treatment device is disposed on the rear back or inside the clothes of a user and the pinch tab is not visible, it is difficult to find the pinch tab and it is thus difficult to easily peel off the electrical treatment device.

Further, since the treatment device main body is heavy, stress is likely to be applied to the pair of conductive adhesive sheets in a direction in which the gravity acts. Thus, depending on the shape of the pair of conductive adhesive sheets, there is a concern that the conductive adhesive sheets may be peeled off from the upper side thereof during treatment.

The present disclosure has been made in view of the problem described above, and an object of the present disclosure is to provide an electrical treatment device that is less likely to peel off during treatment and is easily and entirely removable when removed from a treatment site.

### Solution to Problem

An electrical treatment device according to the present disclosure includes a main body portion including a first outer edge portion and a second outer edge portion facing each other in a first direction in a plan view, a first electrode portion disposed on a side of the first outer edge portion and attachable to a living body of a user, a second electrode portion disposed on a side of the second outer edge portion and attachable to the living body of the user. In a plan view, the first electrode portion and the second electrode portion are spaced apart from each other in the first direction and disposed with at least a central portion of the main body portion located therebetween. The first electrode portion includes a first peeling starting portion serving as a starting point of peeling, and includes a first line located on a side of the second electrode portion. The second electrode portion includes a second peeling starting portion serving as a starting point of peeling, and includes a second line located on the side of the second electrode portion and facing the first line in the first direction. In the electrical treatment device, a gripping portion is provided to cause a peeling force to simultaneously act on the first peeling starting portion and the second peeling starting portion.

According to the above-described configuration, the first electrode portion and the second electrode portion are separated from each other in the first direction and disposed such that at least the central portion of main body portion is located therebetween, and the first line, on the second electrode portion side, included in the first electrode portion and the second line, on the first electrode portion side, included in the second electrode portion face each other in the first direction in which the first outer edge portion and the second outer edge portion of the main body portion face each other. Thus, even when the gravity of the main body portion acts along a direction in which the first line and the second line extend, the first electrode portion and the second electrode portion are less likely to peel off. In addition, even when stress is applied along the first direction from one side and/or the other side of the first direction, since the first electrode portion and the second electrode portion are disposed so as to be separated from each other in the first direction, the first electrode portion and the second electrode portion are less likely to peel off. Accordingly, the electrical treatment device is less likely to peel off from a treatment site during treatment. Since the first electrode portion and the second electrode portion are less likely to peel off due to the stress applied from the direction in which the first line and the second line extend and the stress applied from the first direction, even when an attachment angle of the first electrode portion and the second electrode portion is displaced, the electrical treatment device is less likely to peel off from the treatment site during treatment.

Further, by gripping the gripping portion and moving the gripping portion in a peeling direction, a peeling force can be simultaneously applied to the first peeling starting portion, which serves as a starting point of peeling of the first electrode portion, and the second peeling starting portion, which serves as a starting point of peeling of the second electrode portion. Accordingly, the electrical treatment device can be easily and entirely removed from the treatment site.

In the electrical treatment device according to the present disclosure, the main body portion includes a first end portion located on one side in a second direction intersecting the first direction, and a second end portion located on another side in the second direction. In this case, the first line may be disposed adjacent to the first outer edge portion in a plan view, and the second line may be disposed adjacent to the second outer edge portion in a plan view. The first peeling starting portion may be located at one end of the first line on the one side in the second direction. The second peeling starting portion may be located at one end of the second line on the one side in the second direction. The first peeling starting portion and the second peeling starting portion may be located further to the other side in the second direction than the first end portion. Another end of the first line on the other side in the second direction and another end of the second line on the other side in the second direction may be located further to the other side in the second direction than the second end portion. In this case, the first electrode portion may include a first extending line extending from the other end of the first line toward a first peeling end portion serving as an end point of peeling. The first extending line extends toward the other side in the second direction as the first line extends further to the one side in the first direction. The second electrode portion may include a second extending line extending from the other end of the second line toward a second peeling end portion serving as an end point of peeling. The second extending line extends toward the other side in the second direction as the second line extends further to the other side in the first direction. Further, the gripping portion may be constituted by at least a part of the main body portion.

According to the above-described configuration, when the electrical treatment device is attached to the treatment site with the one side of the second direction facing downward and the other side of the second direction facing upward, the first extending line extending from the other end of the first line toward the first peeling end portion and the second extending line extending from the other end of the second line toward the second peeling end portion can suppress the gravity of the main body portion from concentrating on the other end of the first line and the other end of the second line. Accordingly, the electrical treatment device is less likely to peel off from the treatment site during treatment.

As described above, when the electrical treatment device is attached to the treatment site with the one side in the second direction facing downward and the other side in the second direction facing upward, the first peeling starting portion located at the one end of the first line and the second peeling starting portion located at the one end of the second line are located further to the other side (upper side) in the second direction than the first end portion of the main body portion. Accordingly, when the gripping portion constituted by at least the part of the main body portion is gripped and the main body is moved away from the treatment site, the peeling force can be simultaneously and strongly applied to the first peeling starting portion and the second peeling starting portion. As a result, the electrical treatment device can be removed more easily and entirely.

In the electrical treatment device according to the present disclosure, the main body portion includes a first end portion located on one side in a second direction intersecting the first direction, and a second end portion located on another side in the second direction. In this case, the first line may be disposed adjacent to the first outer edge portion in a plan view, and the second line may be disposed adjacent to the second outer edge portion in a plan view. The first peeling starting portion may be located at one end of the first line on the one side in the second direction. The second peeling starting portion may be located at one end of the second line on the one side in the second direction. The first peeling starting portion and the second peeling starting portion may be located further to the one side in the second direction than the first end portion, and another end of the first line on the other side in the second direction and another end of the second line on the other side in the second direction may be located further to the other side in the second direction than the second end portion. In this case, the first electrode portion may include a first extending line extending from the other end of the first line toward a first peeling end portion serving as an end point of peeling. The first extending line extends toward the other side in the second direction as the first line extends further to the one side in the first direction. The second electrode portion may include a second extending line extending from the other end of the second line toward a second peeling end portion serving as an end point of peeling. The second extending line extends toward the other side in the second direction as the second line extends further to the other side in the first direction. Further, in a plan view, the gripping portion may be provided straddling the first peeling starting portion and the second peeling starting portion on the one side in the second direction with respect to the first end portion. The gripping portion is constituted by a pinch tab portion including a pinch portion on the one side in the second direction with respect to the first peeling starting portion and the second peeling starting portion.

According to the above-described configuration, when the electrical treatment device is attached to the treatment site with the one side in the second direction facing downward and the other side in the second direction facing upward, the first extending line extending from the other end of the first line toward the first peeling end portion and the second extending line extending from the other end of the second line toward the second peeling end portion can suppress the gravity of the main body portion from concentrating on the other end of the first line and the other end of the second line. Accordingly, the electrical treatment device is less likely to peel off from the treatment site during treatment.

Further, as described above, when the electrical treatment device is attached to the treatment site with the one side in the second direction facing downward and the other side in the second direction facing upward, the first peeling starting portion located at the one end of the first line and the second peeling starting portion located at the one end of the second line are located further to the one side (lower side) in the second direction than the first end portion of the main body portion. The pinch tab portion is provided straddling the first peeling starting portion and the second peeling starting portion, and the pinch tab portion includes the pinch portion that can be pinched at a position further to the lower side than the first peeling starting portion and the second peeling starting portion. Thus, by using the main body portion as a guide to find the pinch portion located further to the lower side than the main body portion, and by moving the pinch portion in a direction away from the treatment site, the peeling force can be simultaneously and strongly applied to the first peeling starting portion and the second peeling starting portion. As a result, the electrical treatment device can be removed more easily and entirely.

In the electrical treatment device according to the present disclosure, the main body portion includes a first end portion located on one side in a second direction intersecting the first direction, and a second end portion located on another side in the second direction. In this case, the first line may be disposed adjacent to the first outer edge portion in a plan view, and the second line may be disposed adjacent to the second outer edge portion in a plan view. The first electrode portion may be connected to the first line, and the first electrode portion may include a first linear portion extending linearly on the one side in the second direction with respect to the first end portion. The second electrode portion may be connected to the second line, and the second electrode portion may include a second linear portion extending linearly on the one side in the second direction with respect to the first end portion. In this case, the first linear portion and the second linear portion may be disposed on a same straight line. The first peeling starting portion may be constituted by the first linear portion. The second peeling starting portion may be constituted by the second linear portion. Further, the gripping portion may be constituted by a connection member provided to connect the first linear portion and the second linear portion in a plan view.

According to the above-described configuration, when the electrical treatment device is attached to the treatment site with the one side in the second direction facing downward and the other side in the second direction facing upward, the first linear portion constituting the first peeling starting portion and the second linear portion constituting the second peeling starting portion are connected by the connection member at a position further to the lower side than the main body portion. Thus, by gripping the connection member and moving the connection member in a direction away from the treatment site, the peeling force can be simultaneously applied to the first peeling starting portion and the second peeling starting portion over a wide range. As a result, the electrical treatment device can be removed more easily and entirely.

The electrical treatment device according to the present disclosure may include a first connection portion configured to connect the first line and the main body portion, and a second connection portion configured to connect the second line and the main body portion. In this case, the first line may be located closer to the side of the second outer edge portion than the first outer edge portion in a plan view, and the second line may be located closer to the side of the first outer edge portion than the second outer edge portion in a plan view. The first peeling starting portion may be constituted by the first line. The second peeling starting portion may be constituted by the second line. In this case, the gripping portion may be constituted by the main body portion.

According to the above-described configuration, by gripping the main body portion and moving the main body portion in a direction away from the treatment site, the peeling force can be simultaneously applied to the first line constituting the first peeling starting portion and the second line constituting the second peeling starting portion. As a result, the electrical treatment device can be removed more easily and entirely.

### Advantageous Effects of Invention

According to the present disclosure, it is possible to provide an electrical treatment device that is less likely to be peeled off during treatment and is easily and entirely removable when removed from a treatment site.

### BRIEF DESCRIPTION OF DRAWINGS

Various embodiments are disclosed, by way of example only, with reference to the accompanying schematic drawings in which corresponding reference symbols indicate corresponding parts, in which:

FIG. 1 is a plan view illustrating an electrical treatment device according to a first embodiment;

FIG. 2 is a schematic view of the electrical treatment device according to the first embodiment when viewed from the front;

FIG. 3 is a diagram for describing an operation of removing the electrical treatment device according to the first embodiment;

FIG. 4 is a plan view illustrating an electrical treatment device according to a second embodiment;

FIG. 5 is a plan view illustrating an electrical treatment device according to a third embodiment;

FIG. 6 is a plan view illustrating an electrical treatment device according to a fourth embodiment; and,

FIG. 7 is a schematic view of the electrical treatment device according to the fourth embodiment when viewed from the front.

### DESCRIPTION OF EMBODIMENTS

Embodiments of the present disclosure will be described in detail below with reference to the drawings. Note that, in the following embodiments, identical or common components are given the same reference numerals in the drawings, and descriptions thereof are not repeated.

### First Embodiment

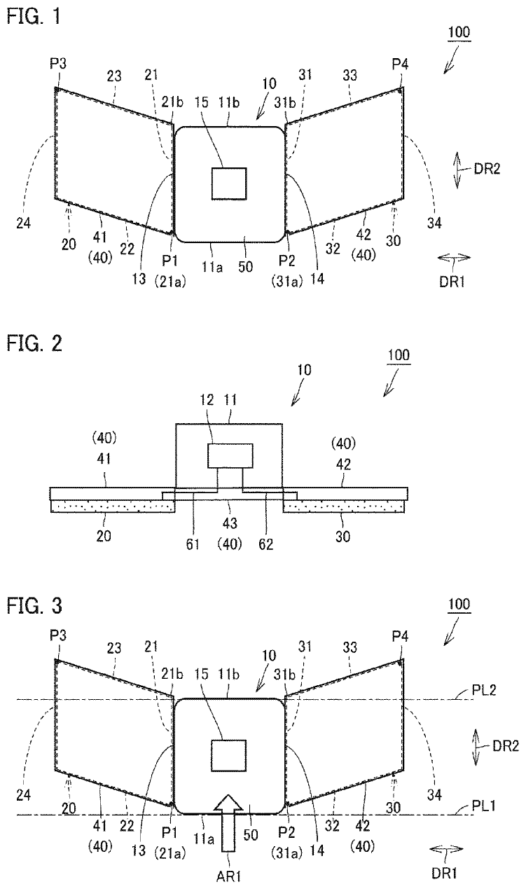

FIG. 1 is a plan view illustrating an electrical treatment device according to a first embodiment. FIG. 2 is a schematic view of the electrical treatment device according to the first embodiment when viewed from the front. An electrical treatment device 100 according to the first embodiment will be described with reference to FIGS. 1 and 2.

As illustrated in FIGS. 1 and 2, the electrical treatment device 100 according to the first embodiment includes a main body portion 10, a first electrode portion 20, a second electrode portion 30, and a pad holding portion 40.

The electrical treatment device 100 is, for example, a low-frequency treatment device that relieves pain of a user by supplying a low-frequency pulse current to the first electrode portion 20 and the second electrode portion 30, which are in contact with a body surface of the user. The frequency of the low-frequency pulse current is, for example, from 1 Hz to 1,200 Hz.

The main body portion 10 includes a housing case 11, a circuit portion 12, and an operation portion 15. The circuit portion 12 is housed in the housing case 11. The circuit portion 12 is electrically connected to the first electrode portion 20 and the second electrode portion 30 via electrical paths 61 and 62. The circuit portion 12 controls the current, the voltage, and the like flowing through the first electrode portion 20 and the second electrode portion 30.

The housing case 11 is provided in a substantially box shape. The housing case 11 has a substantially rectangular shape with rounded corners in a plan view. The housing case 11 includes a first outer edge portion 13 and a second outer edge portion 14 facing each other in a first direction (direction DR1) in a plan view. Note that the shape of the housing case 11 is not limited to the substantially rectangular shape in a plan view, and an appropriate shape such as a circular shape, an elliptical shape, or a trapezoidal shape in a plan view can be selected as long as the housing case 11 includes the first outer edge portion and the second outer edge portion facing each other in the first direction.

The housing case 11 also includes a first end portion 11a and a second end portion 11b. The first end portion 11a and the second end portion 11b face each other in a second direction (direction DR2) intersecting the first direction. The second direction is, for example, a direction orthogonal to the first direction. The first end portion 11a is located on one side in the second direction. The second portion 11b is located on the other side in the second direction.

The operation portion 15 includes, for example, a measurement switch and the like. The operation portion 15 is not limited to being a push type switch and may be, for example, a pressure sensitive type (resistance type) or a proximity type (capacitance type) touch panel type switch. The housing case 11 may include a display portion separately from the operation portion 15.

The first electrode portion 20 and the second electrode portion 30 are spaced apart from each other in the first direction and disposed such that at least a central portion of the main body portion 10 is located between the first electrode portion 20 and the second electrode portion 30 in a plan view.

In the present embodiment, the first electrode portion 20 and the second electrode portion 30 are disposed so as to sandwich the main body portion 10. More specifically, the first electrode portion 20 is disposed on the outer side of the main body portion 10 such that a first line 21 to be described later is adjacent to the first outer edge portion 13 in the first direction. The second electrode portion 30 is disposed on the outer side of the main body portion 10 such that a second line 31 to be described later is adjacent to the second outer edge portion 14 in the first direction.

The first electrode portion 20 and the second electrode portion 30 are electrode portions that cause a current to flow to the user. The first electrode portion 20 and the second electrode portion 30 may be, for example, gel pads or may be laminate bodies constituted by an adhesive layer and an electrode. By supplying a current to the first electrode portion 20 and the second electrode portion 30 in a state in which the first electrode portion 20 and the second electrode portion 30 are in contact with the body surface of the user, it is possible to cause the current to flow to the user.

The first electrode portion 20 has a substantially rhombic shape. The first electrode portion 20 includes the first line 21 and contour lines 22, 23, and 24, which constitute the outer edge of the first electrode portion 20.

The first line 21 is located on the second electrode portion 30 side. The first line 21 extends in a direction intersecting the first direction. The first line 21 extends along the first outer edge portion 13, for example. In the present embodiment, the first line 21 is provided in a linear shape, but is not limited thereto, and may be provided in a zigzag shape or a curved shape.

The first line 21 includes one end 21a and another end 21b. The one end 21a is located on one side in the second direction, and the other end 21b is located on the other side in the second direction. The one end 21a constitutes a first peeling starting portion P1, which serves as a starting point of peeling of the first electrode portion 20.

The contour line 22 is connected to the one end 21a. The contour line 22 extends from the one end 21a toward the other side in the second direction as it extends further to one side in the first direction. Specifically, the contour line 22 is inclined so as to extend toward the other side in the second direction as it extends from the one end 21a further to the one side in the first direction.

The contour line 23 is connected to the other end 21b. The contour line 23 extends from the other end 21b toward the other side in the second direction as it extends further to the one side in the first direction. Specifically, the contour line 23 is inclined so as to extend toward the other side in the second direction as it extends from the other end 21b further to the one side in the first direction. The contour line 23 is parallel to the contour line 22. An end portion, of the contour line 23, located on the side opposite to a side on which the other end 21b is located constitutes a first peeling end portion P3 serving as an end point of peeling. The contour line 23 corresponds to a first extending line extending from the other end 21b of the first line 21 toward the first peeling end portion P3.

The contour line 24 connects the first peeling end portion P3 and the contour line 22. The contour line 24 extends from an end portion, of the contour line 22, located on the side opposite to a side on which the one end 21a is located toward the first peeling end portion P3. The contour line 24 extends along the second direction and is parallel to the first line 21.

The second electrode portion 30 has a substantially rhombic shape. The second electrode portion 30 includes the second line 31 and contour lines 32, 33, and 34, which constitute the outer edge of the second electrode portion 30.

The second line 31 is located on the first electrode portion 20 side. The second line 31 faces the first line 21 in the first direction. The second line 31 extends in a direction intersecting the first direction. The second line 31 extends along the second outer edge portion 14, for example. In the present embodiment, the second line 31 is provided in a linear shape, but is not limited thereto, and may be in a zigzag shape or a curved shape.

The second line 31 includes one end 31a and another end 31b. The one end 31a is located on the one side in the second direction, and the other end 31b is located on the other side in the second direction. The one end 31a constitutes a second peeling starting portion P2, which serves as a starting point of peeling of the second electrode portion 30.

The contour line 32 is connected to the one end 31a. The contour line 32 extends from the one end 31a toward the other side in the second direction as it extends from the one end 31a further to the other side in the first direction. Specifically, the contour line 32 is inclined so as to extend toward the other side in the second direction as it extends from the one end 31a further to the other side in the first direction.

The contour line 33 is connected to the other end 31b. The contour line 33 extends toward the other side in the second direction as it extends from the other end 31b further to the other side in the first direction. Specifically, the contour line 33 is inclined so as to extend toward the other side in the second direction as it extends from the other end 31b further to the other side in the first direction. The contour line 33 is parallel to the contour line 32. An end portion, of the contour line 33, located on the side opposite to a side on which the other end 31b is located constitutes a second peeling end portion P4 serving as an end point of peeling. The contour line 33 corresponds to a second extending line extending from the other end 31b of the second line 31 toward the second peeling end portion P4.

The contour line 34 connects the second peeling end point P4 and the contour line 32. The contour line 34 extends from an end portion, of the contour line 32, located on the side opposite to a side on which the one end 31a is located toward the second peeling end portion P4. The contour line 34 extends along the second direction and is parallel to the second line 31.

The pad holding portion 40 holds the first electrode portion 20 and the second electrode portion 30. The pad holding portion 40 has, for example, flexibility and is provided to be deformable along the body surface. For example, a cloth having insulating properties or the like can be employed as the pad holding portion 40.

The pad holding portion 40 includes a first holding portion 41, a second holding portion 42, and a connection portion 43. The first holding unit 41 holds the first electrode portion 20. The shape of the first holding portion 41 is substantially equal to the shape of the first electrode portion 20. The second holding portion 42 holds the second electrode portion 30. The shape of the second holding portion 42 is substantially the same as the shape of the second electrode portion 30.

The connection portion 43 connects the first holding portion 41 and the second holding portion 42. The main body portion 10 is detachably fixed to the connection portion 43, for example. The connection portion 43 is provided with the electrical paths 61 and 62. In a state in which the main body portion 10 is fixed to the connection portion 43, the first electrode portion 20 and the second electrode portion 30 are electrically connected to the circuit portion 12 by the electrical paths 61 and 62.

When the electrical treatment device 100 is attached to the treatment site with the one side in the second direction facing downward and the other side in the second direction facing upward, the first outer edge portion 13 and the second outer edge portion 14 of the main body portion 10 are parallel to the up-down direction, and the gravity of the main body portion 10 acts on the electrical treatment device 100 toward the one side in the second direction.

Here, in the present embodiment, the first electrode portion 20 and second electrode portion 30 are separated from each other in the first direction and disposed such that at least the central portion of main body portion 10 is located therebetween, and the first line 21, on the second electrode portion 30 side, included in the first electrode portion 20 and the second line 31, on the first electrode portion 20 side, included in the second electrode portion 30 face each other in the first direction in which the first outer edge portion 13 and the second outer edge portion 14 of the main body portion 10 face each other. Thus, even when the gravity of the main body portion 10 acts along a direction in which the first line 21 and the second line 31 extend, the first electrode portion 20 and the second electrode portion 30 are less likely to peel off. In addition, even when stress is applied along the first direction, since the first electrode portion 20 and the second electrode portion 30 are disposed so as to be separated from each other in the first direction, the first electrode portion 20 and the second electrode portion 30 are less likely to peel off. Accordingly, the electrical treatment device 100 is less likely to peel off from the treatment site during treatment. Since the first electrode portion 20 and the second electrode portion 30 are less likely to peel off due to the stress applied from the direction in which the first line 21 and the second line 31 extend and the stress applied from the first direction, even when an attachment angle of the first electrode portion 20 and the second electrode portion 30 is displaced, the electrical treatment device 100 is less likely to peel off from the treatment site during treatment.

Since the first line 21 is disposed adjacent to the first outer edge portion 13 and is provided along the first outer edge portion 13 and the second line 31 is disposed adjacent to the second outer edge portion 14 and is provided along the second outer edge portion 14, the first electrode portion 20 and the second electrode portion 30 can be made even less likely to peel off.

In addition, the contour line 23 (first extending line) extending from the other end 21b of the first line 21 toward the first peeling end portion P3 and the contour line 33 (second extending line) extending from the other end 31b of the second line 31 toward the second peeling end portion P4 can suppress the gravity of the main body portion 10 from concentrating on the other end 21b of the first line 21 and the other end 31b of the second line 31. Accordingly, the electrical treatment device 100 is even less likely to peel off from the treatment site during treatment.

FIG. 3 is a diagram for describing an operation of removing the electrical treatment device according to the first embodiment. An operation of removing the electrical treatment device 100 will be described with reference to FIG. 3.

As illustrated in FIG. 3, the one end 21a of the first line 21 and the one end 31a of the second line 31 are located further to the other side in the second direction than the first end portion 11a. That is, the one ends 21a and 31a are located further to the other side in the second direction than an imaginary line PL1 passing through the first end portion 11a.

The other end 21b of the first line 21 and the other end 31b of the second line 31 are located further to the other side in the second direction than the second end portion 11b. That is, the other ends 21b and 31b are located further to the other side in the second direction than an imaginary line PL2 passing through the second end portion 11b.

When the electrical treatment device 100 is removed, a part of the main body portion 10 on the first end portion 11a side is gripped, and the main body portion 10 is moved toward the other side (that is, the upper side) in the second direction as indicated by an arrow AR1 in FIG. 3. The part of the main body portion 10 on the first end portion 11a side corresponds to a gripping portion 50.

The connection portion 43, of the pad holding portion 40, to which the main body portion 10 is fixed is connected to an end portion, of the first holding portion 41, overlapping the first peeling starting portion P1 and to an end portion, of the second holding portion 42, overlapping the second peeling starting portion P2. Accordingly, by gripping the gripping portion 50 and moving the main body portion 10, a peeling force can be simultaneously applied to the first peeling starting portion P1 and the second peeling starting portion P2. Accordingly, the electrical treatment device can be easily and entirely removed from the treatment site.

Further, a part of the first electrode portion 20 excluding the first peeling starting portion P1 is located further to the other side in the second direction than the first peeling starting portion P1, and a part of the second electrode portion 30 excluding the second peeling starting portion P2 is located further to the other side in the second direction than the second peeling starting portion P2. In addition, since the first extending line (contour line 23) and the second extending line (contour line 33) are provided, a force acting on the other side in the second direction is likely to be directed to the first peeling end portion P3 and the second peeling end portion P4. As a result, the electrical treatment device can be removed more easily and entirely.

### Second Embodiment

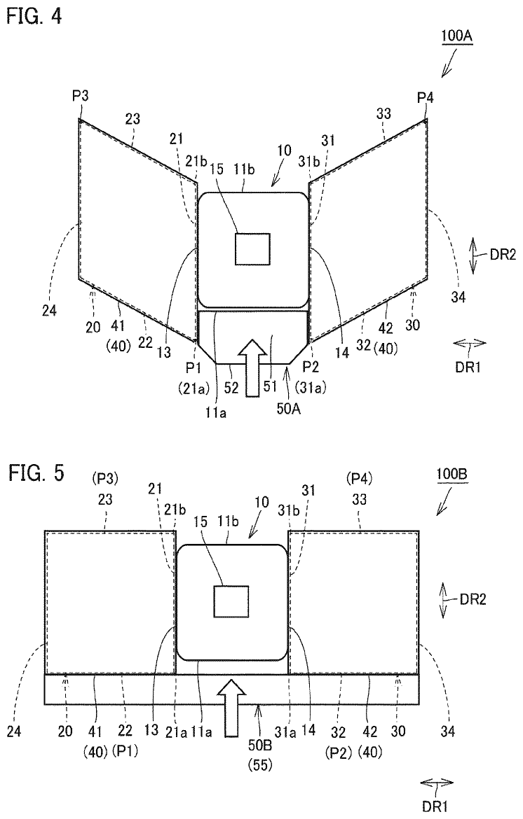

FIG. 4 is a plan view illustrating an electrical treatment device according to a second embodiment. An electrical treatment device 100A according to the second embodiment will be described with reference to FIG. 4.

As illustrated in FIG. 4, when compared with the electrical treatment device 100 according to the first embodiment, the electrical treatment device 100A according to the second embodiment is different in the positions of the first peeling starting portion P1 and the second peeling starting portion P2 and the configuration of the gripping portion 50A. Other configurations are more or less the same.

In the second embodiment, the first electrode portion 20 and the second electrode portion 30 each also have a substantially rhombic shape. In a plan view, the first line 21 of the first electrode portion 20 is disposed adjacent to the first outer edge portion 13 of the main body portion 10, and the second line 31 of the second electrode portion 30 is disposed adjacent to the second outer edge portion 14 of the main body portion 10.

The one end 21a of the first line 21 constitutes the first peeling starting portion P1, and the one end 31a of the second line 31 constitutes the second peeling starting portion P2. The first peeling starting point P1 and the second peeling starting point P2 are located further to the one side in the second direction than the first end portion 11a of the main body portion 10. The other end 21b of the first line 21 and the other end 31b of the second line 31 are located further to the other side in the second direction than the second end portion 11b of the main body portion 10.

The contour line 23 (first extending line) extends from the other end 21b of the first line 21 toward the first peeling end portion P3, and extends toward the other side in the second direction as it extends further to the one side in the first direction. Similarly, the contour line 33 (second extending line) extends from the other end 31b of the second line 31 toward the second peeling end portion P4, and extends toward the other side in the second direction as it extends further to the other side in the first direction.

The gripping portion 50A is constituted by a pinch tab portion 51. The pinch tab portion 51 is located further to the one side in the first direction than the first end portion 11a of the main body portion 10. The pinch tab portion 51 is provided straddling the first peeling starting point P1 and the second peeling starting point P2 in a plan view. The pinch tab portion 51 includes a pinch portion 52 on a leading end side (the side opposite to a side on which the main body portion 10 is located). The pinch portion 52 is located further to the one side in the second direction than the first peeling starting portion P1 and the second peeling starting portion P2.

The pinch tab portion 51 is formed of, for example, a sheet-like member. The pinch tab portion 51 has a desired strength so as not to be torn when the pinch portion 52 is gripped and moved. The pinch tab portion 51 does not include a conductive layer to be attached to the body surface of the user.

In the second embodiment also, when the electrical treatment device 100A is attached to the treatment site with the one side in the second direction facing downward and the other side in the second direction facing upward, the first outer edge portion 13 and the second outer edge portion 14 of the main body portion 10 are parallel to the up-down direction, and the gravity of the main body portion 10 acts on the electrical treatment device 100A toward the one side in the second direction.

In this case also, as in the first embodiment, the first electrode portion 20 and the second electrode portion 30 are separated from each other in the first direction and disposed such that at least the central portion of main body portion 10 is located therebetween, and the first line 21, on the second electrode portion 30 side, included in the first electrode portion 20 and the second line 31, on the first electrode portion 20 side, included in the second electrode portion 30 face each other in the first direction in which the first outer edge portion 13 and the second outer edge portion 14 of the main body portion 10 face each other. Thus, the first electrode portion 20 and the second electrode portion 30 are less likely to peel off, and the electrical treatment device 100A is less likely to peel off from the treatment site during treatment.

In addition, the contour line 23 (first extending line) extending from the other end 21b of the first line 21 toward the first peeling end portion P3 and the contour line 33 (second extending line) extending from the other end 31b of the second line 31 toward the second peeling end portion P4 can suppress the gravity of the main body portion 10 from concentrating on the other end 21b of the first line 21 and the other end 31b of the second line 31. Accordingly, the electrical treatment device 100A is even less likely to peel off from the treatment site during treatment.

When the electrical treatment device 100A is removed, by using the main body portion 10 as a guide to find the pinch portion 52 located further to the lower side (the one side in the first direction) than the main body portion 10, and by moving the pinch portion 52 in a direction away from the treatment site (to the other side in the second direction), the peeling force can be simultaneously and strongly applied to the first peeling starting portion P1 and the second peeling starting portion P2. As a result, the electrical treatment device 100A can be removed more easily and entirely.

### Third Embodiment

FIG. 5 is a plan view illustrating an electrical treatment device according to a third embodiment. An electrical treatment device 100B according to the third embodiment will be described with reference to FIG. 5.

As illustrated in FIG. 5, when compared with the electrical treatment device 100 according to the first embodiment, the electrical treatment device 100B according to the third embodiment is different in the shapes of the first electrode portion 20 and the second electrode portion 30 and the configuration of a gripping portion 50B. Other configurations are more or less the same.

The first electrode portion 20 and the second electrode portion 30 each have a substantially rectangular shape. In a plan view, the first line 21 of the first electrode portion 20 is disposed adjacent to the first outer edge portion 13 of the main body portion 10, and the second line 31 of the second electrode portion 30 is disposed adjacent to the second outer edge portion 14 of the main body portion 10.

The one end 21a of the first line 21 and the one end 31a of the second line 31 are located further to the one side in the second direction than the first end portion 11a of the main body portion 10. The other end 21b of the first line 21 and the other end 31b of the second line 31 are located further to the other side in the second direction than the second end portion 11b of the main body portion 10.

The contour line 22 (first linear portion) is connected to the one end 21a of the first line 21. The contour line 22 extends linearly on the one side in the second direction with respect to the first end portion 11a. The contour line 22 extends substantially parallel to the first direction.

The contour line 23 is connected to the other end 21b of the first line 21. The contour line 23 extends linearly so as to be parallel to the contour line 22 on the other side in the second direction with respect to the second end portion 11b.

The contour line 32 (second linear portion) is connected to the one end 31a of the second line 31. The contour line 32 extends linearly on the one side in the second direction with respect to the first end portion 11a. The contour line 32 extends substantially parallel to the first direction. The contour line 32 is disposed on the same straight line as the contour line 22.

The contour line 33 is connected to the other end 31b of the second line 31. The contour line 33 extends linearly so as to be parallel to the contour line 32 on the other side in the second direction with respect to the second end portion 11b. The contour line 33 is disposed on the same straight line as the contour line 23.

In this case, the first peeling starting portion P1 is constituted by the contour line 22, and the second peeling starting portion P2 is constituted by the contour line 32. The contour line 23 constitutes the first peeling end portion P3, and the contour line 33 constitutes the second peeling end portion P4.

The gripping portion 50B is constituted by a connection member 55 provided so as to entirely connect the contour line 22 and the contour line 32 in a plan view.

The connection member 55 may be provided in a rod shape or a flat shape, for example. The connection member 55 may be formed of a resin member such as plastic, or may be formed of a metal member.

In the third embodiment also, when the electrical treatment device 100B is attached to the treatment site with the one side in the second direction facing downward and the other side in the second direction facing upward, the first outer edge portion 13 and the second outer edge portion 14 of the main body portion 10 are parallel to the up-down direction, and the gravity of the main body portion 10 acts on the electrical treatment device 100B toward the one side in the second direction.

In this case also, as in the first embodiment, the first electrode portion 20 and the second electrode portion 30 are separated from each other in the first direction and disposed such that at least the central portion of main body portion 10 is located therebetween, and the first line 21, on the second electrode portion 30 side, included in the first electrode portion 20 and the second line 31, on the first electrode portion 20 side, included in the second electrode portion 30 face each other in the first direction in which the first outer edge portion 13 and the second outer edge portion 14 of the main body portion 10 face each other. Thus, the first electrode portion 20 and the second electrode portion 30 are less likely to peel off, and the electrical treatment device 100B is less likely to peel off from the treatment site during treatment.

When the electrical treatment device 100B is removed, by using the main body portion 10 as a guide to find the connection member 55 located further to the lower side (the one side in the second direction) than the main body portion 10, and by gripping the connection member 55 and moving the connection member 55 in a direction away from the treatment site (to the other side in the second direction), the peeling force can be simultaneously and strongly applied to the first peeling starting portion P1 and the second peeling starting portion P2 over a wide range. As a result, the electrical treatment device 100B can be removed more easily and entirely.

### Fourth Embodiment

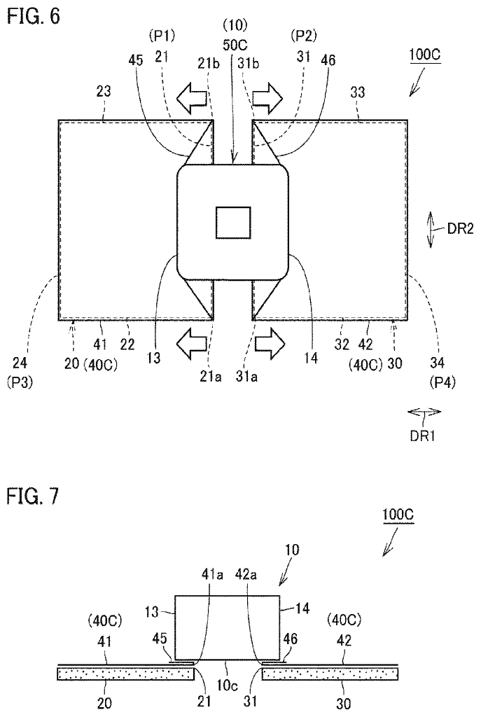

FIG. 6 is a plan view illustrating an electrical treatment device according to a fourth embodiment. FIG. 7 is a schematic view of the electrical treatment device according to the fourth embodiment when viewed from the front. An electrical treatment device 100C according to the fourth embodiment will be described with reference to FIGS. 6 and 7.

As illustrated in FIGS. 6 and 7, when compared with the electrical treatment device 100 according to the first embodiment, the electrical treatment device 100C according to the fourth embodiment is different in the positional relationship between the first and second electrode portions 20 and 30 and the main body portion 10, the configuration of a pad holding portion 40C, and the configuration of a gripping portion 50C.

The first electrode portion 20 and the second electrode portion 30 each have a substantially rectangular shape. In a plan view, the first line 21 of the first electrode portion 20 and the second line 31 of the second electrode portion 30 overlap the main body portion 10. In a plan view, the first line 21 is located further to the second outer edge portion 14 side than the first outer edge portion 13, and the second line 31 is located further to the first outer edge portion 13 side than the second outer edge portion 14.

The pad holding portion 40 includes a first connection portion 45 and a second connection portion 46 instead of the connection portion 43 according to the first embodiment.

The first connection portion 45 connects the first line 21 and the main body portion 10. The first connection portion 45 is a portion that is folded back toward the one side in the first direction from an end portion 41a of the first holding portion 41 located on the main body portion 10 side. The first connection portion 45 is fixed to a portion, of a rear surface 10c of the main body portion 10, located on the first outer edge portion 13 side.

The second connection portion 46 connects the second line 31 and the main body portion 10. The second connection portion 46 is a portion that is folded back toward the other side in the first direction from an end portion 42a of the second holding portion 42 located on the main body portion 10 side. The second connection portion 46 is fixed to a portion, of the rear surface 10c of the main body portion 10, located on the second outer edge portion 14 side.

The first connection portion 45 and the second connection portion 46 are provided with electrical paths (not illustrated), and the first electrode portion 20 and the second electrode portion 30 are electrically connected to the circuit portion 12 via the electrical paths.

In this case, the first line 21 constitutes the first peeling starting portion P1, and the second line 31 constitutes the second peeling starting portion P2. The contour line 24 constitutes the first peeling end portion P3, and the contour line 34 constitutes the second peeling end portion P4.

In the fourth embodiment also, when the electrical treatment device 100C is attached to the treatment site with the one side in the second direction facing downward and the other side in the second direction facing upward, the first outer edge portion 13 and the second outer edge portion 14 of the main body portion 10 are parallel to the up-down direction, and the gravity of the main body portion 10 acts on the electrical treatment device 100C toward the one side in the second direction.

As in the first embodiment, the first electrode portion 20 and the second electrode portion 30 are separated from each other in the first direction and disposed such that at least the central portion of main body portion 10 is located therebetween, and the first line 21, on the second electrode portion 30 side, included in the first electrode portion 20 and the second line 31, on the first electrode portion 20 side, included in the second electrode portion 30 face each other in the first direction in which the first outer edge portion 13 and the second outer edge portion 14 of the main body portion 10 face each other. Thus, the first electrode portion 20 and the second electrode portion 30 are less likely to peel off, and the electrical treatment device 100C is less likely to peel off from the treatment site during treatment. In addition, in the fourth embodiment, since the first electrode portion 20 and the second electrode portion 30 each have a shape in a plan view such that the first electrode portion 20 and the second electrode portion 30 do not easily peel off even as a result of stress from any direction, the degree of freedom of the attachment direction is high.

When the electrical treatment device 100C is removed, by gripping the main body portion 10 and moving the main body portion 10 in a direction away from the treatment site (the normal direction of the rear surface 10c), the peeling force can be simultaneously applied to the first line 21 constituting the first peeling starting portion P1 and the second line 31 constituting the second peeling starting portion P2 as indicated by arrows in FIG. 6. As a result, the electrical treatment device 100C can be removed more easily and entirely.

### Other Modified Examples

In each of the first and second embodiments described above, a case has been described as an example in which the contour lines 23 and 33 are inclined with respect to the first direction, but no such limitation is intended. The contour lines 23 and 33 may be parallel to the first direction. The contour line 23 may be inclined toward the other side in the second direction as it extends further to the one side in the first direction, and the contour line 33 may be inclined toward the other side in the second direction as it extends further to the other side in the first direction. The contour lines 22 and 23 are not limited to being straight lines, and may be curved lines.

In the third embodiment described above, a case has been described as an example in which the contour lines 23 and 33 are parallel to the first direction, but no such limitation is intended. As in the first and second embodiments described above, the contour lines 23 and 33 may be inclined toward the other side in the second direction as it extends further to the outer side in the first direction.

### Supplementary Notes

As described above, the present embodiments include the following disclosures.
Configuration 1: An electrical treatment device (100, 100A, 100B, 100C) includes a main body portion (10) including a first outer edge portion (13) and a second outer edge portion (14) facing each other in a first direction in a plan view, a first electrode portion (20) disposed on a side of the first outer edge portion (13) and attachable to a living body of a user, a second electrode portion (30) disposed on a side of the second outer edge portion (14) and attachable to the living body of the user. In a plan view, the first electrode portion (20) and the second electrode portion (30) are spaced apart from each other in the first direction and disposed with at least a central portion of the main body portion (10) located therebetween. The first electrode portion (20) includes a first peeling starting portion (P1) serving as a starting point of peeling, and includes a first line (21) located on a side of the second electrode portion (30). The second electrode portion (30) includes a second peeling starting portion (P2) serving as a starting point of peeling, and includes a second line (31) located on a side of the first electrode portion (20) and facing the first line (21) in the first direction. A gripping portion (50, 50A, 50B, 50C) is provided to cause a peeling force to simultaneously act on the first peeling starting portion (P1) and the second peeling starting portion (P2).
Configuration 2: In the electrical treatment device (100) according to Configuration 1, the main body portion (10) includes a first end portion (11a) located on one side in a second direction intersecting the first direction, and a second end portion (11b) located on another side in the second direction. The first line (21) is disposed adjacent to the first outer edge portion (13) in a plan view, and the second line (31) is disposed adjacent to the second outer edge portion (14) in a plan view. The first peeling starting portion (P1) is located at one end (21a) of the first line (21) on the one side in the second direction, and the second peeling starting portion (P2) is located at one end (31a) of the second line (31) on the one side in the second direction. The first peeling starting portion (P1) and the second peeling starting portion (P2) are located further to the other side in the second direction than the first end portion (11a). Another end (21b) of the first line (21) on the other side in the second direction and another end (31b) of the second line (31) on the other side in the second direction are located further to the other side in the second direction than the second end portion (11b). The first electrode portion (20) includes a first extending line (23) extending from the other end (21b) of the first line (21) toward a first peeling end portion (P3) serving as an end point of peeling, the first extending line (23) extending toward the other side in the second direction as the first line (21) extends further to one side in the first direction. The second electrode portion (30) includes a second extending line (33) extending from the other end (31b) of the second line (31) toward a second peeling end portion (P4) serving as an end point of peeling, the second extending line (33) extending toward the other side in the second direction as the second line (310 extends further to another side in the first direction. The gripping portion (50) is constituted by at least a part of the main body portion (10).
Configuration 3: In the electrical treatment device (100A) according to Configuration 1, the main body portion (10) includes a first end portion (11a) located on one side in a second direction intersecting the first direction, and a second end portion (11b) located on another side in the second direction. The first line (21) is disposed adjacent to the first outer edge portion (13) in a plan view, and the second line (31) is disposed adjacent to the second outer edge portion (13) in a plan view. The first peeling starting portion (P1) is located at one end (21a) of the first line (21) on the one side in the second direction, and the second peeling starting portion (P2) is located at one end (31a) of the second line (31) on the one side in the second direction. The first peeling starting portion (P1) and the second peeling starting portion (P2) are located further to the one side in the second direction than the first end portion (11a). Another end (21b) of the first line (21) on the other side in the second direction and another end (31b) of the second line (31) on the other side in the second direction are located further to the other side in the second direction than the second end portion (11b). The first electrode portion (20) includes a first extending line (23) extending from the other end (21b) of the first line (21) toward a first peeling end portion (P3) serving as an end point of peeling, the first extending line (23) extending toward the other side in the second direction as the first line (21) extends further to the one side in the first direction. The second electrode portion (30) includes a second extending line (33) extending from the other end (31b) of the second line (31) toward a second peeling end portion (P4) serving as an end point of peeling, the second extending line (33) extending toward the other side in the second direction as the second line (31) extends further to the other side in the first direction. In a plan view, the gripping portion (50A) is provided straddling the first peeling starting portion (P1) and the second peeling starting portion (P2) on the one side in the second direction with respect to the first end portion (11a), the gripping portion (50A) being constituted by a pinch tab portion (51) including a pinch portion (52) on the one side in the second direction with respect to the first peeling starting portion (P1) and the second peeling starting portion (P2).
Configuration 4: In the electrical treatment device (100B) according to Configuration 1, the main body portion (10) includes a first end portion (11a) located on one side in a second direction intersecting the first direction, and a second end portion (11b) located on another side in the second direction. The first line (21) is disposed adjacent to the first outer edge portion (13) in a plan view, and the second line (31) is disposed adjacent to the second outer edge portion (14) in a plan view. The first electrode portion (20) is connected to the first line (21), the first electrode portion (20) including a first linear portion (22) extending linearly on the one side in the second direction with respect to the first end portion (11a). The second electrode portion (30) is connected to the second line (31), the second electrode portion (30) including a second linear portion (32) extending linearly on the one side in the second direction with respect to the first end portion (11a). The first linear portion (22) and the second linear portion (32) are disposed on a same straight line. The first peeling starting portion (P1) is constituted by the first linear portion (22), and the second peeling starting portion (P2) is constituted by the second linear portion (32). The gripping portion (50B) is constituted by a connection member (55) provided to connect the first linear portion (22) and the second linear portion (32) in a plan view.
Configuration 5: The electrical treatment device (100C) according to Configuration 1 includes a first connection portion (45) configured to connect the first line (21) and the main body portion (10, and a second connection portion (46) configured to connect the second line (31) and the main body portion (10). The first line (21) is located closer to the side of the second outer edge portion (14) than the first outer edge portion (13) in a plan view, and the second line (31) is located closer to the side of the first outer edge portion (13) than the second outer edge portion (14) in a plan view. The first peeling starting portion (P1) is constituted by the first line (21), and the second peeling starting portion (P2) is constituted by the second line (31). The gripping portion (50C) is constituted by the main body portion (10).

The embodiments disclosed herein are illustrative in all respects and are not intended as limitations. The scope of the present invention is indicated by the claims and includes all meaning equivalent to the claims and changes within the scope of the claims.

### REFERENCE NUMERALS LIST

10 Main body portion, 10c Rear surface, 11 Housing case, 11a First end portion, 11b Second end portion, 12 Circuit portion, 13 First outer edge portion, 14 Second outer edge portion, 15 Operation portion, 20 First electrode portion, 21 First line, 21a One end, 21b Another end, 22, 23, 24 Contour line, 30 Second electrode portion, 31 Second line, 31a One end, 31b Another end, 32, 33, 34 Contour line, 40, 40C Pad holding portion, 41 First holding portion, 41a, 42a End portion, 42 Second holding portion, 43 Connection portion, 45 First connection portion, 46 Second connection portion, 50, 50A, 50B, 50C Gripping portion, 51 Pinch tab portion, 52 Pinch portion, 55 Connection member, 61, 62 Electrical path, 100, 100A, 100B, 100C Electrical treatment device, P1 First peeling starting portion, P2 Second peeling starting portion, P3 First peeling end portion, P4 Second peeling end portion, PL1, PL2 Imaginary line

What is claimed is:

1. An electrical treatment device comprising:
a main body portion including a first outer edge portion and a second outer edge portion facing each other in a first direction in a plan view;
a first electrode portion disposed on a side of the first outer edge portion and attachable to a living body of a user;
a second electrode portion disposed on a side of the second outer edge portion and attachable to the living body of the user, wherein:
in a plan view, the first electrode portion and the second electrode portion are spaced apart from each other in the first direction and disposed with at least a central portion of the main body portion located therebetween,
the main body includes a circuit portion configured to control a current flowing through the first electrode portion and the second electrode portion, and a housing case configured to accommodate the circuit portion,
the first electrode portion includes a first peeling starting portion serving as a starting point of peeling, and includes a first line located on a side of the second electrode portion,
the first line is disposed adjacent to the first outer edge portion in a plan view,
the second line is disposed adjacent to the second outer edge portion in a plan view,
the second electrode portion includes a second peeling starting portion serving as a starting point of peeling, and includes a second line located on a side of the first electrode portion and facing the first line in the first direction,
a gripping portion is provided to cause a peeling force to simultaneously act on the first peeling starting portion and the second peeling starting portion,
the main body portion includes a first end portion located on one side in a second direction intersecting the first direction, and a second end portion located on another side in the second direction,
in an attached state in which the first electrode portion and the second electrode portion are attached with the one side in the second direction facing downward and the other side in the second direction facing upward, the first peeling starting portion is located at one end of the first line on the one side in the second direction, and the second peeling starting portion is located at one end of the second line on the one side in the second direction,
the first peeling starting portion and the second peeling starting portion are located further to the other side in the second direction than the first end portion,
another end of the first line on the other side in the second direction and another end of the second line on the other side in the second direction are located further to the other side in the second direction than the second end portion,
the first electrode portion includes, in the attached state, a first extending line extending from the other end of the first line toward a first peeling end portion serving as an end point of peeling, the first extending line extending toward the other side in the second direction as the first line extends further to one side in the first direction,
the second electrode portion includes, in the attached state, a second extending line extending from the other end of the second line toward a second peeling end portion serving as an end point of peeling, the second extending line extending toward the other side in the second direction as the second line extends further to another side in the first direction, and
the gripping portion is constituted by at least a part of the main body portion.

2. An electrical treatment device comprising:
a main body portion including a first outer edge portion and a second outer edge portion facing each other in a first direction in a plan view;
a first electrode portion disposed on a side of the first outer edge portion and attachable to a living body of a user;
a second electrode portion disposed on a side of the second outer edge portion and attachable to the living body of the user, wherein:
in a plan view, the first electrode portion and the second electrode portion are spaced apart from each other in the first direction and disposed with at least a central portion of the main body portion located therebetween,
the main body includes a circuit portion configured to control a current flowing through the first electrode portion and the second electrode portion, and a housing case configured to accommodate the circuit portion,
the first electrode portion includes a first peeling starting portion serving as a starting point of peeling, and includes a first line located on a side of the second electrode portion,
the second electrode portion includes a second peeling starting portion serving as a starting point of peeling, and includes a second line located on a side of the first electrode portion and facing the first line in the first direction,
the first line is disposed adjacent to the first outer edge portion in a plan view,
the second line is disposed adjacent to the second outer edge portion in a plan view,
a gripping portion is provided to cause a peeling force to simultaneously act on the first peeling starting portion and the second peeling starting portion,
the main body portion includes a first end portion located on one side in a second direction intersecting the first direction, and a second end portion located on another side in the second direction,
in an attached state in which the first electrode portion and the second electrode portion are attached with the one side in the second direction facing downward and the other side in the second direction facing upward, the first peeling starting portion is located at one end of the first line on the one side in the second direction, and the second peeling starting portion is located at one end of the second line on the one side in the second direction,
the first peeling starting portion and the second peeling starting portion are located further to the one side in the second direction than the first end portion,
another end of the first line on the other side in the second direction and another end of the second line on the other side in the second direction are located further to the other side in the second direction than the second end portion,
the first electrode portion includes, in the attached state, a first extending line extending from the other end of the first line toward a first peeling end portion serving as an end point of peeling, the first extending line extending toward the other side in the second direction as the first line extends further to the one side in the first direction,
the second electrode portion includes, in the attached state, a second extending line extending from the other end of the second line toward a second peeling end portion serving as an end point of peeling, the second extending line extending toward the other side in the second direction as the second line extends further to the other side in the first direction, and
in a plan view, the gripping portion is provided straddling the first peeling starting portion and the second peeling starting portion on the one side in the second direction with respect to the first end portion, the gripping portion being constituted by a pinch tab portion including a pinch portion on the one side in the second direction with respect to the first peeling starting portion and the second peeling starting portion.

3. An electrical treatment device comprising:
a main body portion including a first outer edge portion and a second outer edge portion facing each other in a first direction in a plan view;
a first electrode portion disposed on a side of the first outer edge portion and attachable to a living body of a user;
a second electrode portion disposed on a side of the second outer edge portion and attachable to the living body of the user, wherein:
in a plan view, the first electrode portion and the second electrode portion are spaced apart from each other in the first direction and disposed with at least a central portion of the main body portion located therebetween,
the main body includes a circuit portion configured to control a current flowing through the first electrode portion and the second electrode portion, and a housing case configured to accommodate the circuit portion,
the first electrode portion includes a first peeling starting portion serving as a starting point of peeling, and includes a first line located on a side of the second electrode portion,
the second electrode portion includes a second peeling starting portion serving as a starting point of peeling, and includes a second line located on a side of the first electrode portion and facing the first line in the first direction,
the first line is disposed adjacent to the first outer edge portion in a plan view,
the second line is disposed adjacent to the second outer edge portion in a plan view,
a gripping portion is provided to cause a peeling force to simultaneously act on the first peeling starting portion and the second peeling starting portion,
the main body portion includes a first end portion located on one side in a second direction intersecting the first direction, and a second end portion located on another side in the second direction,
the first electrode portion is connected to the first line, the first electrode portion including a first linear portion extending linearly on the one side in the second direction with respect to the first end portion,
the second electrode portion is connected to the second line, the second electrode portion including a second linear portion extending linearly on the one side in the second direction with respect to the first end portion,
the first linear portion and the second linear portion are disposed on a same straight line,
the first peeling starting portion is constituted by the first linear portion,
the second peeling starting portion is constituted by the second linear portion,
the gripping portion is constituted by a connection member provided to connect the first linear portion and the second linear portion in a plan view, and
the connection member is constituted by a rod-shaped member.

4. An electrical treatment device comprising:
a main body portion including a first outer edge portion and a second outer edge portion facing each other in a first direction in a plan view;
a first electrode portion disposed on a side of the first outer edge portion and attachable to a living body of a user;
a second electrode portion disposed on a side of the second outer edge portion and attachable to the living body of the user;
a first connection portion configured to connect the first line and the main body portion; and
a second connection portion configured to connect the second line and the main body portion, wherein:
in a plan view, the first electrode portion and the second electrode portion are spaced apart from each other in the first direction and disposed with at least a central portion of the main body portion located therebetween,
the first electrode portion includes a first peeling starting portion serving as a starting point of peeling, and includes a first line located on a side of the second electrode portion,
the second electrode portion includes a second peeling starting portion serving as a starting point of peeling, and includes a second line located on a side of the first electrode portion and facing the first line in the first direction,
a gripping portion is provided to cause a peeling force to simultaneously act on the first peeling starting portion and the second peeling starting portion,
the first line is located closer to the side of the second outer edge portion than the first outer edge portion in a plan view,
the second line is located closer to the side of the first outer edge portion than the second outer edge portion in a plan view,
the first peeling starting portion is constituted by the first line,
the second peeling starting portion is constituted by the second line, and
the gripping portion is constituted by the main body portion.
